(19) United States
(12) Patent Application Publication
(10) Pub. No.: US 2012/0101402 A1
(43) Pub. Date: Apr. 26, 2012

(54) METHOD AND SYSTEM FOR CONTROLLING A DEVICE

(75) Inventor: Hung Tan Nguyen, New South Wales (AU)

(73) Assignee: UNIVERSITY OF TECHNOLOGY, SYDNEY, Ultimo (AU)

(21) Appl. No.: 13/265,389

(22) PCT Filed: Apr. 20, 2010
(86) PCT No.: PCT/AU2010/000444

§ 371 Date: Jan. 18, 2012

(30) Foreign Application Priority Data
Apr. 21, 2009 (AU) 2009901716

(51) Int. Cl. A61B 5/0402; G06N 3/02; G06N 5/00

(52) U.S. Cl. 600/544; 706/45; 706/20

(57) ABSTRACT

A method for controlling a device, the method including the steps of, in a processing system: receiving a signal associated with a thought pattern (100), the signal being received from a single electroencephalogram (EEG) channel, the EEG channel having sensed any one or a combination of a visual cortex, a parietal cortex, or the area in between the visual cortex and the parietal cortex, and generating a control signal based on the determined thought pattern (120), the control signal being configured to initiate control of the device.

### FIELD OF THE INVENTION

The present invention relates to a method and system for determining/sensing thought signals or thought patterns and processing these signals in order to control a device. In particular, the present invention relates to a method and system for controlling devices such as wheelchairs, automobiles, and processing systems.

### DESCRIPTION OF THE BACKGROUND ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Severe disability, especially in the case of spinal cord injuries (SCI) can cost many billions of dollars each year in direct and indirect expenditure, not including the suffering and emotional stress involved. It is estimated that worldwide there are more than 670,000 people living with SCI, and that the cost of managing the care of SCI patients is approaching $4 billion in the US and $11 billion worldwide (WinterGreen Research 2007).

In one particular example, for people living with SCI, wheelchairs have made some tasks more accessible. In this example, for people with disability such as SCI, being able to master wheelchair skills can be the difference between dependence and independence in daily life. However, not all people with disabilities have the dexterity to control even the joystick on a powered wheelchair. The lack of physical coordination to control a moving powered wheelchair with the added problem of unexpected obstacles and driving errors can lead to accidents. Importantly, just operating the vehicle can trigger intense physical and mental fatigue.

Furthermore, some disabilities, such as tetraplegia SCI, stroke and muscular dystrophy, require the use of complete hands-free assistive technologies.

Additionally, there are many other actions, such as for example, performing an action like moving an object, driving a car, etc, which are either difficult or impossible for people with SCI.

Thus, there is required a system and/or method for controlling a device, such as a wheelchair, automobile, or another type of processing system, which overcomes, at least ameliorates one or more disadvantages of existing arrangements, or provides an alternative to existing arrangements.

### SUMMARY OF THE PRESENT INVENTION

According to a first broad form, there is provided a method for controlling a device, the method including the steps of, in a processing system: receiving a signal associated with a thought pattern; and, generating a control signal based on the determined thought pattern, the control signal being configured to initiate control of the device.

In one particular example, the method includes determining the thought pattern based on the received signal.

According to one example, the method includes receiving the signal from a single electroencephalogram (EEG) channel derived from sensing an area of the brain.

In a further example, the single EEG channel senses any one or a combination of visual cortex, parietal cortex, or the area in between the visual cortex and the parietal cortex.

In yet another example, the single EEG channel is positioned away from the motor cortex.

According to another aspect, the thought pattern is associated with an action that is to be performed in relation to the device.

In yet a further form, determining the thought pattern includes analysing the signal received and classifying the signal received.

In accordance with another example, analysing the signal received includes: transforming EEG data from the signal into the frequency domain using a discrete Fast Fourier Transform; and, dividing the transformed data into Delta, Theta, Alpha, Beta, and Gamma frequency bands.

In a further example, classifying the signal includes using a neural network classification system.

According to another example, the neural network has an optimal number of hidden nodes based on the highest level of Bayesian evidence.

In another aspect, the method includes receiving a second signal, the second signal being associated with a mental or emotional state.

In a further form, the method includes using the second signal to complement the signal associated with the thought command pattern.

According to a further aspect, the method includes receiving information associated with physical surroundings; the information received being used to monitor the generated control signal.

In yet another aspect, the method includes analysing and classifying the signal in real-time.

According to another example, the signal associated with the thought pattern is received from a wireless transmitter positioned around a user's head.

In yet a further example, the device includes any one or a combination of a wheelchair, an automobile, a game console, and another processing system.

According to a second broad form, there is provided herein a processing system for controlling a device, the processing system being configured to: receive a signal associated with a thought pattern; determine the thought pattern based on the received signal; and, generate a control signal based on the determined thought pattern, the control signal being configured to initiate control of the device.

### BRIEF DESCRIPTION OF THE DRAWINGS

An example of the present invention will now be described with reference to the accompanying drawings, in which:—

FIG. 1 is a flow diagram of an example method/process that can be utilised to embody or give effect to a particular embodiment;

FIG. 2 is schematic diagram of an example of the method/process of FIG. 1, in use;

FIG. 3 is a functional block diagram of an example processing system that can be utilised to embody or give effect to a particular embodiment;

FIG. 4 is a flow diagram of an example method/process that can be utilised to embody or give effect to another particular embodiment;

FIG. 5 is a photograph of an example wheelchair which may be used with the system described herein;

FIGS. 6A is a schematic diagram of an example of the system described herein as a part of a headband placed around a user's head. FIG. 6A shows an example back view of the user's head.

FIG. 6B is a schematic diagram of an example of the system described herein as a part of a headband placed around a user's head. FIG. 6B shows an example top view of the user's head.

FIG. 7 is a schematic diagram of an example of an electrode which can be used with the system described herein.

### DETAILED DESCRIPTION INCLUDING EXAMPLE MODES

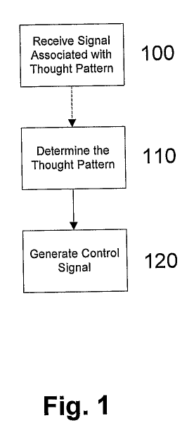

An example of a method/process for controlling a device will now be described with reference to FIG. 1.

In particular, FIG. 1 shows that the method can include the steps of receiving a signal associated with a thought pattern at step 100, optionally determining the thought pattern at step 110, and generating a control signal at step 120. In this particular example, the control signal can be configured to initiate control of the device.

According to one particular example, the signal is received from a single electroencephalogram (EEG) channel derived from sensing an area of the brain. It will be appreciated by persons skilled in the art that a single EEG channel can allow for control of the device to occur in real-time. Furthermore, a single channel can be provided with limited noise and a clear signal.

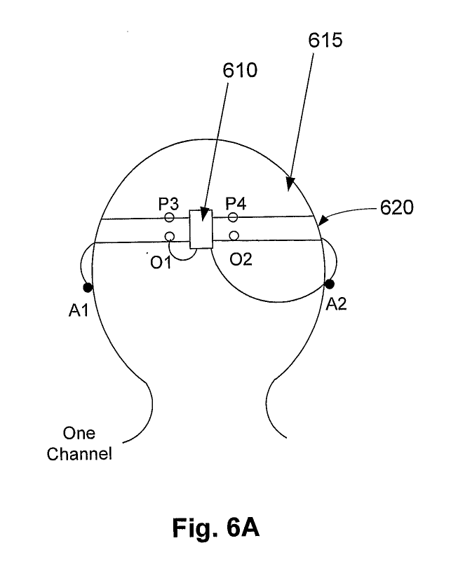

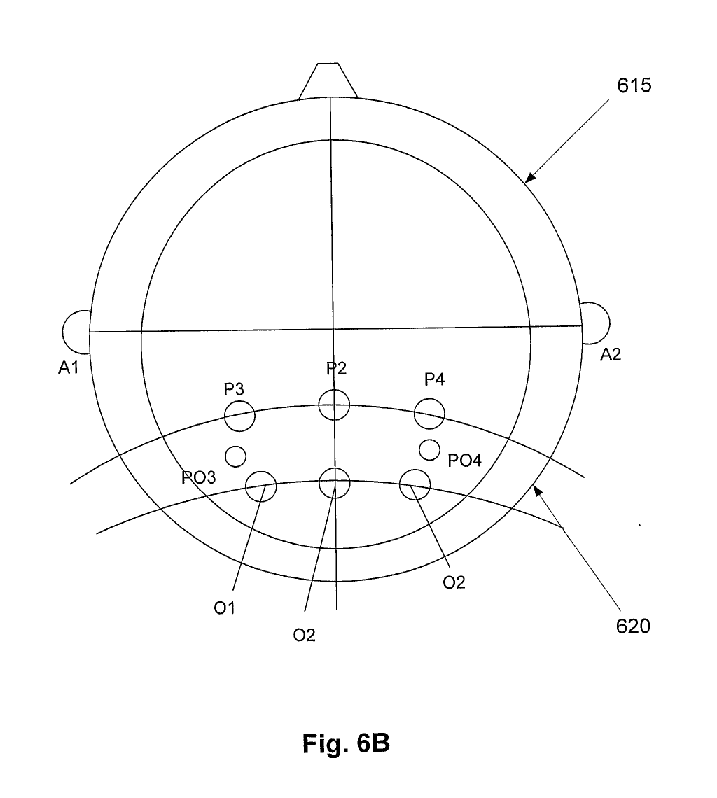

In one particular example, the single EEG channel is positioned on an area of a person's head, such that the area sensed in on or near the visual cortex or parietal cortex as shown in FIG. 6B, and according to a further example, the channel is placed away from the motor cortex. Thus, by sensing the visual (or occipital cortex), the system described herein can determine/analyse alpha, beta, and theta waves in order to then determine the thought pattern. The parietal cortex can then be used to attenuate the signal received from the visual cortex, by determining an emotional state, which can aid in the control of the device.

According to a further example, the thought pattern is associated with an action that is to be performed in relation to the device, and not necessarily by actual movement of a person. Thus, for example, the thought pattern can be imagining a particular task in the mind which can be associated to a respective action that is to be performed by the device.

Thus, for example, a device, such as a wheelchair or the like, may be controlled by a processing system receiving a signal associated with a thought pattern, where the thought pattern is associated with a thought of a user of the wheelchair. The thought can include, for example, solving a simple arithmetic problem, or imagining a formation of a letter, which could be associated with wanting the wheelchair to move in a particular direction. The sensed signal from the parietal cortex can then be used to determine an emotional state of the user, and thus further control the wheelchair. For example, if the wheelchair is moving too fast, and the user becomes scared or nervous, the wheelchair control can be managed accordingly.

Accordingly, the processing system can determine the thought pattern depending on the signal received, and control the wheelchair accordingly.

Determining the thought pattern can include analysing the signal received and classifying the signal received. Thus, for example, analysing the signal received can include transforming EEG data from the signal into the frequency domain using a discrete Fast Fourier Transform, and dividing the transformed data into delta, theta, alpha, beta, and gamma frequency bands. Furthermore, classifying the signal can include using a neural network classification system, where the neural network has an optimal number of hidden nodes based on the highest level of Bayesian evidence. This is further discussed below. Notably, analysing and classifying the signal received can occur in real-time.

The method described herein can also include receiving a second signal, the second signal being associated with a mental or emotional state, where the second signal can be used to normalise, improve, or complement the signal associated with the thought pattern. Notably the second signal may be received from the occipital region, parietal region, or a region there between. It will also be appreciated that the second signal is not necessarily required and can be used to activate some safety measures, which may also be detectable from the first signal.

Furthermore, the method described herein can also include receiving information associated with physical surroundings, the information received being used to monitor and/or manage the generated control signal.

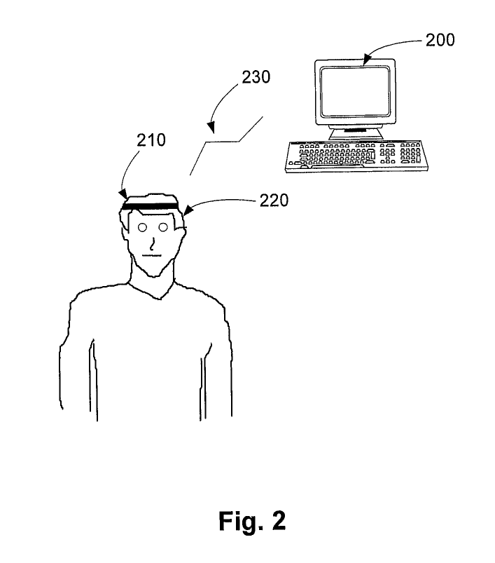

As shown in FIG. 2, the process of FIG. 1 can be performed using a processing system 200, which is configured to be able to communicate with a transmitter 210 positioned around a user's head 220. In this particular example, the signal associated with the thought pattern is received via wireless communication 230 between the transmitter 210 and the processing system 200. The processing system 200 may then be able to initiate control of the device, such as a wheelchair or the like. This can be by either communicating with an embedded processing system of the device, or the processing system 200 itself being embedded in the device.

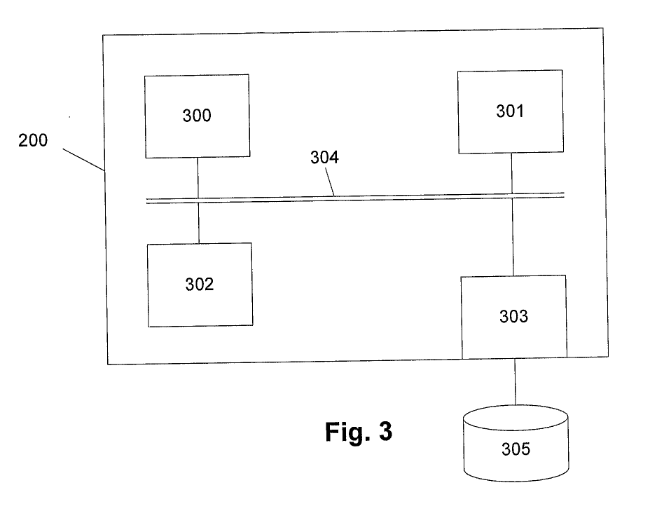

It will be appreciated by persons skilled in the art that the processing system 200 may be any form of processing system and may even be integrated with the transmitter 210. Accordingly, any form of suitable processing system 200 may be used. An example is shown in FIG. 3. In this example, the processing system 200 includes at least a processor 300, a memory 301, an input/output (I/O) device 302, such as a keyboard, and display, and an external interface 303, coupled together via a bus 304 as shown. Notably, the memory 301 can include a database, or the processing system may be configured to communicate with an external data store 305.

Thus, it will be appreciated that the processing system 200 may be formed from any suitable processing system, embedded processing system (as a part of the device), or the like, such as a microcontroller system, programmable PC, lap-top, hand-held PC, smart phone, or the like, which is typically operating applications software to enable data transfer which can then be used to control systems such as wheelchairs, automobiles and other processing systems. According to one particular example, the processing system 200 can be a small embedded receiver, which can function as a part of a stand-alone real-time system as a part of a device such as a wheelchair, or the like.

According to yet a further example, it will also be appreciated that the processing system 200 may form a part of a network such as the Internet, a LAN, or WAN, or a part of any distributed architecture, where the network can be used to collect information from several wheelchairs or devices and administer and monitor the control of these devices.

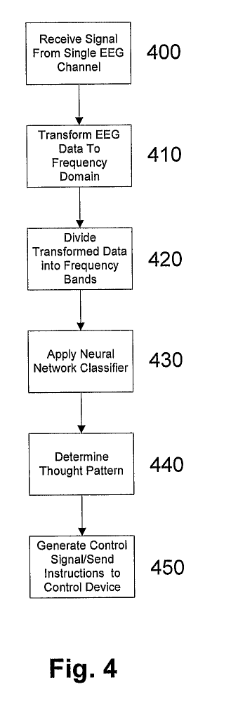

A further example of the process for controlling a device will now be described in more detail with respect to FIG. 4.

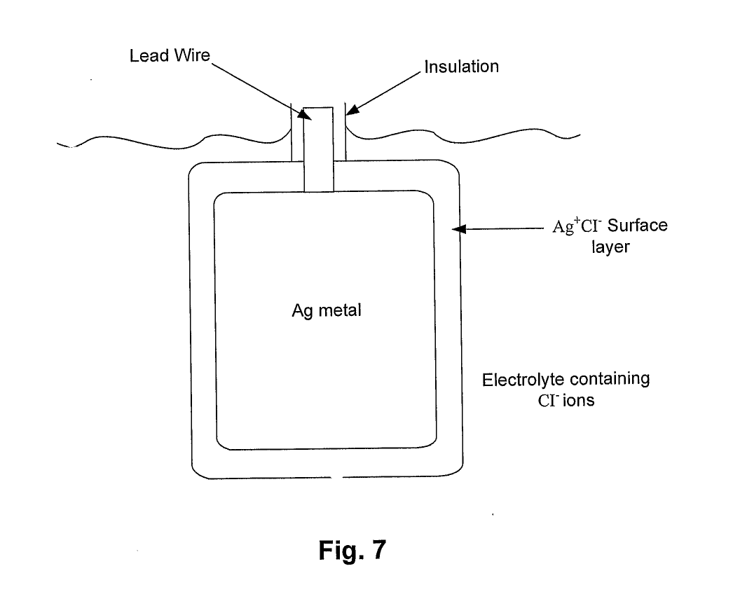

In this example, at step 400, the processing system 200 can receive a signal from a single EEG channel which is typically placed at a particular location (as shown in FIG. 7B and usually away from the motor cortex) of a user's head. At step 410, the processing system 200 can transform the received EEG data into the frequency domain, and at step 420 the transformed data can be divided into various frequency bands. At step 430 a neural network classifier can be applied to the full spectrum or the frequency bands, and the thought pattern which caused the particular EEG signal at step 400 can be determined at step 440.

At step 450 the processing system 200 can then generate a control signal and/or send an instruction, or the like, in order to control a device such as a wheelchair or the like, depending on the thought pattern which generated the EEG signal at step 400.

Accordingly, there is provided herein a method and system which can be used to identify and classify thought patterns and to use these to control various devices. In one particular example, the classification of thought patterns is processed with sufficient speed and accuracy to control the device without extended delays between the generation of the thought pattern by the user and the instruction being implemented by the device that is being controlled. According to another example, the thought pattern obtained from the user can be processed by just one EEG channel.

Notably, whilst the method and system described herein has many applications, the real-time nature of the system makes the system and method described herein particularly suitable as a means of directing a vehicle, such as a wheelchair. It will also be appreciated that the device can include any one or a combination of a wheelchair, an automobile, a game console, and/or any other processing system or machine.

Further examples for the system and method of controlling a device are described below.

### Further Examples

According to one particular example, a method of classifying thought patterns in real-time can include the steps of receiving signals from one EEG channel only for each task and classifying thought patterns based on the received signals. The main EEG input/sensor may be positioned on the visual cortex (such as O1 or O2) or parietal cortex (such as P3 or P4) as described in the standard International 10-20 System of EEG electrode placement. Any EEG site in between (such as PO3 or PO4) as in the modified expanded 10-20 system may also be used. Note that the reference input of this EEG channel may be located on one of the earlobes (A1 or A2) or any of the other main EEG sites.

In this particular example, one single differential amplifier is required with only one active EEG electrode to be sensed at any given time from a site located on the visual cortex, parietal cortex or motor cortex.

According to a further example, there is provided herein a method of controlling a vehicle including the steps of classifying thought patterns by way of the method/system described herein, and controlling the vehicle based on the real-time classification of the thought patterns.

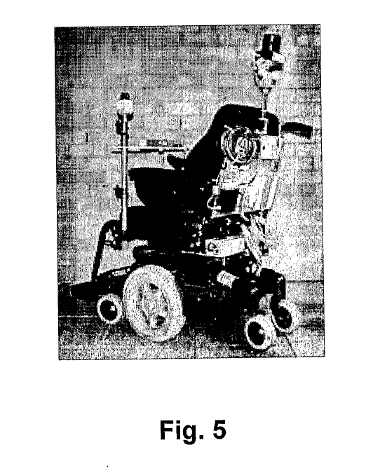

Thus, for example, the vehicle may be a power wheelchair, as shown in FIG. 5, and the classification of thought patterns may include thought patterns that are classified as representing commands to drive the vehicle forward, to turn left, to turn right or to stop the vehicle. The thought patterns may be produced by a user visualising a (F)igure being rotated about an axis to go forward, mentally composing a (L)etter to go left, solving a simple a(R)ithmetic problem to go right or clo(S)ing his/her eyes to stop the vehicle. Thus, a user may mentally compose a letter in order to cause a particular action, such as wheelchair movement. Notably, this is described only as a guide, as any thought pattern can be associated with a specific action.

According to yet a further example, there is also provided herein a method of controlling a vehicle including the steps of receiving signals from a user interface device, classifying the received signals using a neural network with an optimal number of hidden nodes based on the highest level of Bayesian evidence, and controlling the vehicle based on the classification of the signals.

For example, two Bayesian neural networks can be developed for real-time identification of the user's intention and physiological states. Notably, Bayesian neural networks were firstly introduced as a practical and powerful means to improve the generalisation of neural networks. The training of a Bayesian neural network adjusts weight decay parameters automatically to optimal values for the best generalisation by estimating the evidence for each model and no separate validation set is required. In evidence framework, the Gaussian assumptions are used to approximate the posterior distribution of weights and biases. The regularisation is undertaken to prevent any weights becoming excessively large, which can cause poor generalisation. In particular, for multi-layer perceptron neural network classifiers with G different groups of weights and biases, the “weight decay” is added to the data error function E_{D} in order to obtain the objective function in the form:

$$S = {E}_{D} + {\sum }_{g = 1}^{G} {ξ}_{g} {E}_{{W}_{g}} , \\ {E}_{D} = - {\sum }_{n = 1}^{N} \{ {t}^{( n )} ln {z}^{( n )} + ( 1 - {t}^{( n )} ) ln ( 1 - {z}^{( n )} ) \} , \\ {E}_{{W}_{g}} = \frac{1}{2} {‖ {w}_{g} ‖}^{2} \qquad ( 1 )$$

where E_{D} is the “cross-entropy” data error function and E_{W}_{_{g}}(g=1, . . . , G) are weight functions corresponding to weight and bias groups, ξ_{g} are “non-negative” scalars, sometimes called hyperparameters for controlling the distributions of weights and biases in different groups and w_{g} is the vector of weights or biases in the gth group. The evidence of a two-layer network Xᵢ with M hidden nodes is given by:

$$\begin{matrix}LogEv ( {X}_{i} ) = - {E}_{D} ( w ) + ln Occ ( w ) + {\sum }_{g = 1}^{G} ln Occ ( {ξ}_{g} ) & ( 2 ) \\ ln Occ ( w ) = - {\sum }_{g = 1}^{G} {ξ}_{g}^{MP} {E}_{{W}_{g}}^{MP} + {\sum }_{g = 1}^{G} \frac{{W}_{g}}{2} ln {ξ}_{g} - \frac{1}{2} ln \vert A \vert + ln M ! + M ln 2 , \\ ln Occ ( {ξ}_{g} ) = \frac{1}{2} ln ( \frac{4 π}{{γ}_{g}^{MP}} ) + K & ( 3 )\end{matrix}$$

where W_{g} is the number of weights and biases in the gth group and K is a constant. The best network will be selected with the highest log evidence.

For each identification task (intention and physiological state), the evidence framework for Bayesian inference to the training set and the required number of hidden nodes for the optimal neural network architecture can be found when it yields the highest evidence. Advanced adaptive optimal Bayesian neural-network classification algorithms can be obtained to recognise the intention and physiological state of the operator in real-time. It would be possible to allow the system to learn as it adapts to gains experience about the thought patterns of a particular operator. In particular, it would do so even when the severely disabled operator experiences thought pattern variations due to many reasons, including deterioration in his/her disability state. Thus, the algorithm described herein may be developed to adapt to and receive commands from each individual user, and can be trained for each individual.

According to one example, this can be performed by running a calibration test when necessary and can allow the Bayesian neural network classify algorithm to be updated in real-time accordingly. It should be noted that similar results can be achieved using other machine learning techniques (such as genetic algorithms or standard neural works), but this would usually require a time-consuming trial-and-error process to reach an optimal architecture. On the other hand, the described Bayesian neural network strategy can provide an automatic process to obtain an optimal neural network through its evidence framework (that is, equation (2) above is the log evidence equation, an optimal neural network has a highest log evidence value).

In this particular example, the EEG data for each location (for example O1 and P4) is transformed into the frequency domain using a discrete Fast Fourier Transform (FFT) and is then broken up into the delta, theta, alpha, beta and gamma frequency bands. All or part of this frequency spectrum is used as one or more inputs to the classification feed-forward neural network. For example, it is expected that the final may be mostly based upon information from theta, alpha and beta bands. The optimal number of hidden nodes is based on the highest level of evidence. The network has four output nodes, each of which corresponds to one mental command.

The method and system described herein can provide an effective brain computer interface or brain computer interaction that operates effectively in real-time without significant time delay due to acquiring, processing and classifying the signal using just one EEG channel.

Notably, it is also possible to use the same EEG sensor or an independent signal from another EEG sensor to provide additional information beyond the determination of the command for the wheelchair. For example, the signal may provide the mental or emotional state of the user (such as the effect/influence of fatigue or stress/anxiety on the user and the signal received from the first electrode). This information may be used to activate a particular safety aspect of the vehicle.

It will also be appreciated that further information, supplemental to the EEG sensor signal may be used to effect control of a device, such as a wheelchair. Accordingly, information associated with physical surroundings of the device/user can be received, where the information is used to monitor the generated control signal.

Thus, for example, obstacle avoidance and wheelchair guidance (through doorways and turns) can be integrated and implemented into a powered wheelchair using cameras, encoders, laser-based and neuro-sliding strategies. Accordingly, a separate system may monitor the user's commands and intervene if an intended action can cause harm or danger to the user (such as the wheelchair being directed towards and obstacle).

Notably, although a Bayesian neural network has been described as an example classifier herein, it will be appreciated by persons skilled in the art that any other form of classification system which is deemed suitable may be used within the system/method described herein. Furthermore, the classification system may be used to adapt the system for a particular user of the system, depending on that particular user's disability. Thus for example, the system/method may associate a particular user's thought patterns with actions and may adapt accordingly.

According to yet a further example, a wireless EEG system has been developed, which includes a Tx transmitter head set module that is typically worn by a user, and a Rx transceiver module. In the transmitter module, there are two differential EEG channels, a PIC micro-controller and a 2.4 GHz transceiver with a telemetry range of 10 m. The Rx transceiver module consists of a 2.4 GHz transceiver for two channels, a PIC micro-controller and USB communication to a PC. The USB receiver uses a virtual COM port for transfer to the PC (Mac mini, 2 GHz Intel Core 2 Duo, 2 GB, 80 GB hard drive) with the baud rate fixed at 115,200 bps. A small embedded microcontroller system can be used in place of the PC which can be used to classify intended commands accurately in real-time.

As shown in FIGS. 6A and 6B, the wireless system can include a wireless ECG amplifier 610, which can be located on a headband 620 that is used to secure the EEG sensors (as indicated at P2, P3, P4, O1, O2, A1, A2) in place, on a user's head 615.

As discussed herein, the system and method discussed can be used to control many devices, and in one particular example, can be used to control a wheelchair. A wheelchair which can be used to implement the system and method described herein is the M1 Roller Chair (which is an example of a type of wheelchair). The features of the M1 Roller Chair include a midwheel drive motor, which gives the wheelchair the ability to turn on the spot, which makes control with more control schemes more feasible.

In order to control the M1 Roller wheelchair in real-time, the method described herein was implemented in computer program code, and in one particular example, software was written under a Windows(R) environment. Software was written in both National Instruments CVI and C programming language to allow the analysis of the EEG signal. Training of the neural network classifier was undertaken using back propagation techniques with various learning rules (delta learning rule, conjugate gradient, etc.). A total of 300 samples from 5 people were collected for the preliminary training of the classifier. Using the Bayesian neural network framework, these samples were divided into two sets: a training/validation set and a test set with 150 samples each. Following the completion of the training, the network was analysed using the test set. From this preliminary analysis, an effective overall accuracy was achieved in the test set for the four different commands.

Notably, it is possible that with an adaptive learning strategy, the eventual system may be able to achieve a high overall accuracy. According to one particular example, the EEG sensor is an active electrode (Ag—AgCl) which can be held in place by a headband. To enable better electrical contact with the scalp, a high conductivity gel can be applied to the scalp underneath the electrode.

An example structure of an Ag—AgCl electrode is shown in FIG. 7. A silver metal base with attached insulated lead wire is coated with a layer of the ionic compound AgCl. AgCl remains stable as it is slightly soluble in water. The electrode is then immersed in an electrolytic bath in which the principal anion of the electrolyte is Cl—. Notably, the EEG sensor could be of the type which does not require gel.

Thus, it will be appreciated that the system/method described herein may be used to provide hands-free mobility assistance for severely disabled people (power wheelchairs, environmental control units, etc.). Furthermore, it may also be used to provide effective real-time control strategies in a semi-autonomous wheelchair to provide mobility assistance. It may be used in conjunction with embedded autonomous technology to assist the user in performing task-specific navigation and allow the human-machine system to operate safely.

Thus, the use of the technology described herein may promote independence by enabling people with disabilities to perform tasks they may not otherwise be able to accomplish. Furthermore, the use of one EEG signal allows for a system which can provide real-time mobility in, which is time efficient (that is, has a relatively fast response time).

It will also be appreciated that there are many other uses for the method and system described herein, including, and not limited to real-time control of vehicles including public transportation, or effecting control of a vehicle in the case of an emergency (such as a driver heart attack, for example). The device may also be used for controlling a game console or for entertaining industry, and is not necessarily limited to people living with spinal cord injury (SCI). In one particular example, the system and method described herein may be used to provide remote control of a device over the Internet, or at another geographical location.

The foregoing describes only some embodiments of the present invention, and modifications and/or changes can be made thereto without departing from the scope and spirit of the invention, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

1. A method for controlling a device, the method including the steps of, in a processing system:
a) receiving a signal associated with a thought pattern, the signal being received from a single electroencephalogram (EEG) channel, the EEG channel having sensed any one or a combination of a visual cortex, a parietal cortex, or the area in between the visual cortex and the parietal cortex; and,
b) generating a control signal based on the determined thought pattern, the control signal being configured to initiate control of the device.

2. The method of claim 1, wherein the method includes positioning the single EEG channel away from the motor cortex.

3. The method of claim 1, wherein the method includes positioning the EEG channel on user's scalp.

4. The method of claim 1, wherein the thought pattern is associated with an action that is to be performed in relation to the device.

5. The method of claim 4, wherein the action is to move the device.

6. The method of claim 1, wherein the method further includes determining the thought pattern by analysing the signal received and classifying the signal received.

7. The method of claim 6, wherein analysing the signal received includes:
a) transforming EEG data from the signal into the frequency domain using a discrete Fast Fourier Transform; and,
b) dividing the transformed data into delta, theta, alpha, beta, and gamma frequency bands.

8. The method of claim 6, wherein classifying the signal includes using a neural network classification system.

9. The method of claim 8, wherein the neural network has an optimal number of hidden nodes based on the highest level of Bayesian evidence.

10. The method of claim 1, wherein the method includes receiving a second signal, the second signal being associated with a mental or emotional state.

11. The method of claim 10, wherein the method includes using the second signal to complement the signal associated with the thought pattern.

12. The method of claim 1, wherein the method includes receiving information associated with physical surroundings, the information received being used to monitor and/or manage the generated control signal.

13. The method of claim 1, wherein the method includes analysing and classifying the signal in real-time.

14. The method of claim 1, wherein the method includes receiving the signal associated with the thought pattern from a wireless transmitter positioned around a user's head.

15. The method of claim 1, wherein the device includes any one or a combination of a wheelchair, an automobile, a game console, and another processing system.

16. A processing system for controlling a device, the processing system being configured to:
a) receive a signal associated with a thought pattern, the signal being received from a single electroencephalogram (EEG) channel, the EEG channel having sensed any one or a combination of a visual cortex, a parietal cortex, or the area in between the visual cortex and the parietal cortex; and;
b) generate a control signal based on the determined thought pattern, the control signal being configured to initiate control of the device.

17. The processing system of claim 16, the processing system being configured to perform a method for controlling a device, the method including the steps of, in a processing system:
a) receiving a signal associated with a thought pattern, the signal being received from a single electroencephalogram (EEG) channel, the EEG channel having sensed any one or a combination of a visual cortex, a parietal cortex, or the area in between the visual cortex and the parietal cortex; and,
b) generating a control signal based on the determined thought pattern, the control signal being configured to initiate control of the device.
